(19) United States
(12) Patent Application Publication
(10) Pub. No.: US 2008/0107707 A1
(43) Pub. Date: May 8, 2008

(54) POLYMERIZABLE ANTIMICROBIAL COMPOSITION

(75) Inventors: McKinley C. Lawson, Boulder, CO (US); Kristi S. Anseth, Boulder, CO (US)

Correspondence Address: Don D. Cha, 547 Buena Vista Road, Golden, CO, 80401, US

(73) Assignee: REGENTS OF THE UNIVERSITY OF COLORADO, Boulder, CO (US)

(21) Appl. No.: 11/774,047

(22) Filed: Jul. 6, 2007

Related U.S. Application Data
(60) Provisional application No. 60/806,678, filed on Jul. 6, 2006.

(51) Int. Cl. A61K 9/00; A61K 38/02; A61K 31/397; A61K 38/43; A61P 31/00; A61K 38/48; A61F 13/00; A61L 15/16

(52) U.S. Cl. 424/423; 514/8; 514/210.05; 514/210.15; 424/94.1; 424/94.67; 424/429; 424/446

(57) ABSTRACT

The present invention provides a polymerizable antimicrobial composition and a method for using the same. The polymerizable antimicrobial composition of the invention comprises an antimicrobial compound, a linker, and a polymerizable function group.

### CROSS-REFERENCE TO RELATED APPLICATIONS

This application claims the priority benefit of U.S. Provisional Application No. 60/806,678, filed Jul. 6, 2006, which is incorporated herein by reference in its entirety.

### STATEMENT REGARDING FEDERALLY FUNDED RESEARCH

The U.S. Government has a paid-up license in this invention and the right in limited circumstances to require the patent owner to license others on reasonable terms as provided for by the terms of Grant No. EEC-0444771 awarded by the National Science Foundation.

### FIELD OF THE INVENTION

The invention relates to a polymerizable antimicrobial composition comprising an antimicrobial compound that is linked or attached to a polymerizable moiety.

### BACKGROUND OF THE INVENTION

Infection following the implantation of orthopedic hardware remains a serious and expensive complication. It is estimated that more than one million hip replacements are done each year worldwide. Modern surgical practice has reduced the incidence of infection following total hip arthroplasty to approximately 1-2% in the general population. The percentage is thought to be higher for other joint replacements because of their proximity to the skin surface and the more limited experience in joint design. Those patients with periprosthetic joint infection must undergo lengthy antibiotic therapy and quite often surgical revision. Antibiotic therapy alone is widely accepted as being ineffective and is considered only when concomitant illness precludes surgery. Two-stage operations in which contaminated hardware is removed and temporarily replaced with antibiotic-loaded spacers are common. A second operation is performed several weeks after the first to implant new, definitive hardware. One-stage operations are sometimes used, but two-stage techniques are considered the standard, at least in the United States. Problems including loss of viable bone stock, long periods of hospitalization, severe functional impairment, recurrent infection, and less than ideal treatment options still present definite challenges to both surgeon and patient.

The physiologic location of orthopedic infections makes them inherently difficult to treat due to poor antibiotic penetration into bone and joint spaces and the formation of bacterial biofilms. A biofilm is defined as “a microbially derived sessile community characterized by cells that are irreversibly attached to a substratum or interface or to each other, are imbedded in a matrix of polymeric substances that they have produced, and exhibit an altered phenotype with respect to growth rate and gene transcription.” Biofilms can form on virtually any surface including sequestra of dead bone and implant surfaces alike. Diffusion limitations, adaptive stress responses, down-regulated rates of cell division of the deeply embedded microbes, and changes in bacterial gene expression profiles all contribute to the difficulty in eliminating biofilm-forming organisms.

The concentrations of antibiotics required for bactericidal activity against sessile organisms can be several orders of magnitude higher than for planktonic bacteria—the rule of thumb being 1000-1500 times higher. This places a premium on extensive wound irrigation and debridement at the time of surgery.

However, it is difficult to physically remove all organisms, and recurrent infection may be problematic days, months, or even years after the initial infection. Biofilm infections usually persist until surgically removed, even in individuals with competent innate and adaptive immune responses. Therapeutic strategies aimed at enzymatically degrading biofilm matrix polymers, blocking biofilm matrix synthesis, and interfering with cell-to-cell communication have all been suggested. Specialty surface coatings that interfere with biofilm primary adhesion (“docking”) or secondary anchoring (“locking”) are also potentially useful.

Numerous materials including antibiotic-loaded bone cement and antibiotic loaded poly(methyl methacrylate) beads have been used in the treatment of orthopedic infections. Such bone cements have been in use for more than three decades and are becoming the standard of practice in Europe and Scandinavia for primary and revision hip and knee arthroplasty. Of note is that protocols for use of antibiotic-loaded bone cements have not been universally standardized. Before May, 2003, there were no FDA approved antibiotic-loaded bone cements in the United States, leaving surgeons to mix their own formulations in the operating suite. The currently approved FDA products are of the low dose variety and are likely best suited for prophylaxis rather than treatment. There are no FDA approved products containing multiple antibacterial agents.

Antibiotic-loaded bone cements are quite good at delivering high local levels of an antibacterial agent for a short period of time, usually a few days, and, when loaded at high dose (>2 g antibiotic/40 g cement), may provide effective antibiotic levels in surrounding tissues for several months. However, high-dose loading can lead to altered polymer network architecture and subsequent mechanical compromise of bone cements. Moreover, antibiotic-loaded poly(methyl methacrylate) will continue to elute therapeutic agents for weeks or months at suboptimal levels. When these levels are below the breakpoint sensitivity limit—defined as the antimicrobial level that marks the transition between susceptibility and induction of antibiotic resistance—one may worry about promoting the development of antibiotic-resistant organisms in situ.

It has been noted that bone cement has an optimal surface for colonization, and, though antibiotic loading can yield reductions in biofilm formation, organisms are still able to grow on gentamicin-loaded bone cements, for example. Therefore, there is a continuing need for antibacterial surface modification of bone cements. A system having continual surface activity, well-controlled release properties when applicable, and the ability to avoid cement mechanical weakness is particularly desirable.

Various new materials for treating and preventing orthopaedic infections have been described. Many of these designs involve novel biomaterials and ways of loading those materials with pharmaceuticals. For example, it is possible to construct antibiotic-loaded beads or other spatially complex devices from copolymers of poly(lactic-co-glycolic) acid such that they degrade and release entrapped compounds for extended periods of time. It is also conceivable that biodegradable bone cements may be constructed in an analogous fashion.

Other approaches include polymerizing various multifunctional monomers or macromers around implant devices to form cross-linked coatings and loading said coatings with elutable species. Implants have also been coated with degradable polymers by solvent casting techniques. The coatings can be loaded with various antibiotics to provide a controlled release system. Antibacterial polymers have been described that are in some sense “bioresponsive.”One example involves a polymer in which the backbone incorporates a fluoroquinolone antibiotic that must be cleaved from the polymer for activity. This approach relies on antibiotics having two appropriate functional groups so as to properly incorporate into the polymer backbone. The antibacterial is released in vivo through the action of host enzymes or non-enzymatic hydrolysis or oxidation. The polymer is not per se antibacterial, but released fragments are. Since fluoroquinolones must be internalized by bacteria, improper cleavage fragments would be anticipated to show much decreased activity over the parent drug(s). One would only expect small cleavage fragments to be properly internalized. Also, some of the monomers of this type are not water soluble, and reported antibacterial doses more than one thousand times those of the unmodified species are required. It might be preferable to have a polymeric antibacterial that does not require internalization, that does not require cleavage for activity, and that has comparable activity to its parent drug.

Limited examples also exist of polymer compositions that possess antimicrobial properties independent of chemical elution or cleavage. These polymers are often not based on traditional antibiotic species, and, in some cases, modes of action are not well defined. Questions remain when determining where these materials would be most appropriately used clinically. Furthermore, convenient ways of attaching these materials to relevant medical articles may not be available. A platform utilizing antibacterials with known toxicity, known mode of action, and well-demonstrated clinical efficacy might be advantageous.

Recently, covalent attachment of an antibiotic to a titanium surface has been achieved, where a monolayer of a modified form of the antibiotic vancomycin was attached to titanium particles. When mixed with bacterial suspensions, these particles killed viable organisms. However, this approach is potentially limited by the small amount of antibiotic that is deposited since it provides only monolayer surface coverage.

A technique of attaching a monolayer of an antimicrobial peptide to a flat titanium substrate has also been reported to kill bacteria in solution. The peptide is bound to a poly(ethylene glycol) (PEG) tether which then attaches to the metal surface. The tether provides mobility for the peptide. This method, like the aforementioned, appears to be limited by the relatively small amount of antibacterial agent that can be attached to the surface. Furthermore, since antibacterial peptides must assume specific secondary and tertiary structures to maintain activity, this coating may not be amenable to common storage processes (i.e. protein denaturation is a concern).

There is also a question of whether monolayer-type coatings, in general, will be of ample physical integrity to withstand implantation in certain settings. For example, a significant shear is experienced by the femoral component of a total hip replacement upon insertion into the intramedullary canal of the femur. Whether or not ample antibacterial material would remain attached is questionable. A preferable coating would have long-term physical stability and have multiple layers of active antibacterial constituent(s) such that simply removing the outermost molecules would not render the coating inactive.

Accordingly, there is a need for polymerizable antimicrobial compositions that can be attached to a substrate surface without the limitation of being a monolayer coating.

### SUMMARY OF THE INVENTION

One aspect of the present invention provides an antimicrobial composition that comprises a polymerizable functional group that is linked to an antimicrobial compound. In some embodiments, the polymerizable functional group is attached to the antimicrobial compound through a linker moiety. In this manner, a wide variety of antimicrobial compounds can be used in the present invention.

In one particular embodiment, the antimicrobial composition is of the formula:

V-L-A Formula I

where
V is a polymerizable functional group, typically an olefin, that is covalently linked to L, or a moiety comprising such polymerizable functional group;
L is a linker comprising from about 5 to about 500 linking chain atoms; and
A is an antimicrobial compound that is covalently linked to L.

Typically, any antimicrobial compound that can be covalently linked to a linking moiety can be used in the invention. Generally, such antimicrobial compounds comprise one or more functional groups that allow formation of at least one covalent bond between the antimicrobial compound and the linking moiety. In some embodiments, the antimicrobial compound is linked to a linker (i.e., linking moiety) via an amide bond.

Within this embodiment, in some instances the antimicrobial compound is an antibacterial compound. Suitable antibacterial compounds include, but are not limited to, vancomycin and other natural, synthetic, or semi-synthetic glycopeptide antibiotics (teicoplanin, ramoplanin, decaplanin, oritavancin, dalbavancin, ramoplanin, THRX-1179, etc.), bacitracin, cephalexin, cefadroxil, cefaclor, cefotaxime, cefprozil, loracarbef, ceforanide, cefepime, cefibutin, cefdinir, cefditorin pivoxel, ceftizoxime, ceftazidime, ceftriaxone, ceftazidime, cephradine, cefixime, aztreonam, amoxicillin, penicillamine, ampicillin, or a mixture thereof. The antimicrobial compound can also be an enzyme such as lysostaphin.

In some embodiments, the linker, L, is polyethylene glycol (multifunctional or linear), polylactic acid, polyglycolic acid, poly(lactic-co-glycolic) acid, polyurethane, polyester, polypeptide, or a combination thereof.

In one particular embodiment, the antimicrobial compositions is vancomycin-polyethylene glycol-acrylate (VPA). A specific embodiment of the antimicrobial compositions of the invention include vancomycin-PEG(3400)-acrylate.

Another aspect of the present invention provides a method for forming a surface-bound antimicrobial composition on a substrate. Such a method generally includes:
providing a substrate having a surface-bound polymerizable functional group;
contacting the substrate surface with an antimicrobial composition that comprises a polymerizable functional group that is linked to an antimicrobial compound
forming a covalent bond between the surface-bound polymerizable functional group and the polymerizable olefin functional group to form a surface-bound antimicrobial substrate.

In some embodiments, the antimicrobial composition is of Formula I.

Antimicrobial compositions of the invention can be bound to a surface of a various medical products and medical devices.

In some instances, formation of the covalent bond between the surface-bound polymerizable functional group and the polymerizable olefin functional group comprises photopolymerization. In other instances such covalent bond formation comprises radical polymerization. Still in other instances, such covalent bond formation comprises living radical polymerization.

In other embodiments, the step of forming the covalent bond can include encapsulating a bioactive material within the polymer matrix of surface-bound antimicrobial substrate. In this manner, a wide variety of combination therapy and/or prophylactic treatment can be achieved simultaneously.

In some embodiments, the medical product is bone cement, polymeric bone graft, synthetic tissue scaffold, wound dressing, tissue adhesive, a medical device, or a dental composite including, but not limited to, filling materials and implantable hardware.

Still in other embodiments, the medical device is a catheter, an intravenous tube, a stent, a vascular graft, a nasogastric tube, a contact lens, orthopedic hardware, such as but not limited to a total hip replacement, a total knee replacement, an intramedullary rod, a pin, a screw, a nail, a wire, or a plate.

Yet in other embodiments, the antimicrobial composition is covalently bound to the surface of said medical device. In other embodiments, the antimicrobial composition is covalently bound to said medical product. It should be appreciated that the antimicrobial composition can be attached directly to a surface or polymerized as part of a bulk polymeric material (e.g., medical polymer).

Still another aspect of the present invention provides a method of coating a medical device with an antimicrobial compound of the invention. In this aspect of the invention, the method includes:
contacting the medical device surface comprising a polymerizable functional group with the antimicrobial composition to produce a surface-coated medical device; and
polymerizing the surface-coated medical device thereby producing covalent linkage between the polymerizable functional group of the medical device surface and the polymerizable olefin functional group of the antimicrobial composition.

### BRIEF DESCRIPTION OF THE DRAWINGS

FIG. 1 is a schematic illustration of photochemical attachment of VPA to Silanized Ti-6Al-4V alloy;

FIG. 2 illustrates a method for synthesizing vancomycin-PEG(3400)-acrylate (VPA);

FIG. 3 is a gel filtration chromatogram of VPA-PEG(3400)-acrylate using a Sephadex G-25F column, 33 cm×2.5 cm (height×i.d.), deionized H₂O mobile phase, ca. 3 mL/min volume flow rate;

FIG. 4 is a 500 MHz ¹H NMR spectrum of vancomycin-PEG(3400)-acrylate in D₂O;

FIGS. 5A and 5B show PEG(375)-monoacrylate Coated Ti-6Al-4V Disc;

FIGS. 6A-6C show results of elution controls for VPA Copolymer Coatings;

FIGS. 7A and 7B show antibacterial surface activity of VPA copolymer coating; and

FIG. 8 is chemical structure of THRX-1179.

### DETAILED DESCRIPTION OF THE INVENTION

Compositions and methods of the invention provide various polymerizable antimicrobial monomers that maintain antimicrobial action before and after a polymerization reaction. Antimicrobial compositions of the invention can be attached to a substrate surface via any suitable polymerization reaction including, but not limited to, a radical polymerization reaction initiated by a thermal activatable initiator, a visible light or long wavelength ultraviolet light-activatable initiator, benzoyl peroxide, potassium persulfate, ammonium persulfate, or other known free-radical initiator, or a photochemical polymerization reaction, as well as other known polymerization reaction such as living radical polymerization, anionic or cationic polymerization, or Michael addition polymerization.

Compositions of the invention can be used to coat a surface of a variety of substrates including, but not limited to, Ti-6Al-4V orthopedic alloy or titanium (e.g., with VPA). In some embodiments, the resulting coating is covalently bonded to the substrate surface, resistant to delamination, and/or provides multiple antimicrobial (e.g., vancomycin) molecules that are attached to a linker (e.g., PEG) of desired length and pendant to each polymeric backbone chain (e.g., polyacrylate).

Compositions and methods of the invention provide a significant advantage over monolayer coverage strategies in terms of the number of molecules available for interaction with microbials (e.g., bacterial cells). Without being bound by any theory, it is believed that the resulting polymer inhibits and/or kills bacteria through a contact mediated mechanism.

It is believed that vancomycin's activity derives from its ability to bind the D-ALA-D-ALA terminal end of bacterial peptidoglycan precursors and thus interfere with peptidoglycan crosslinking in the cell wall. Accordingly, internalization of vancomycin by bacteria is not required for its bacteriacidal activity. By linking vancomycin to a polymer backbone via a poly(ethylene glycol) tether, it is believed that adequate mobility is afforded for significant interaction with bacterial cells, and that vancomycin need not be cleaved from the polymer for activity. It should be appreciated that while some of the examples described herein demonstrate a PEG linker of ca. 3,400 molecular weight, other PEG chain-lengths can also be used.

One material often used in orthopedic implants is an alloy of Ti-6Al-4V (by weight 90% Ti, 6% Al, 4% V). This material has a native oxide layer. In some embodiments of the invention, the surface of this material is further oxidized, e.g., by appropriate treatment with a solution of hydrogen peroxide and sulfuric acid. The oxide thus formed has a number of available hydroxyl groups which can be reacted with a silane reagent having a desired terminal functionality. The silane forms a self-assembled monolayer over the oxide surface. In some instances, methacryloxypropyltrimethoxysilane was used because inter alia it provides an ethylenically unsaturated double bond in the form of a methacrylate group to or from which polymer chains can be grafted.

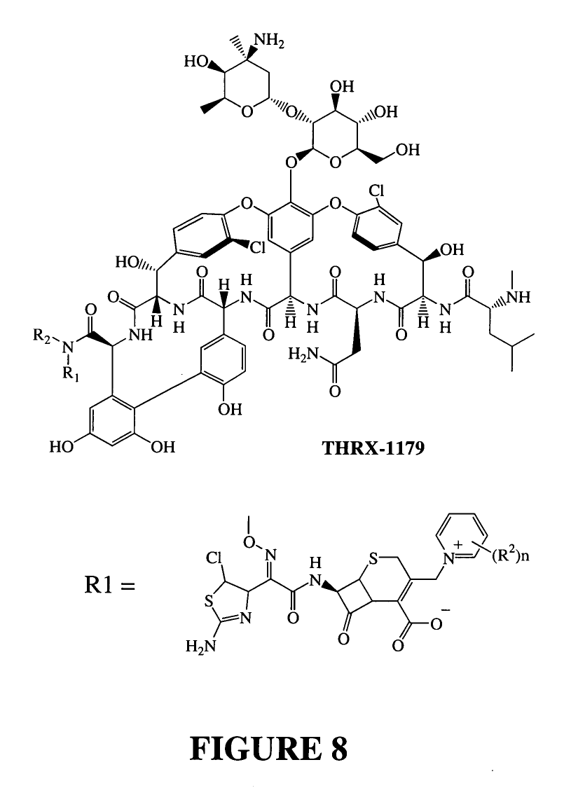

It should be appreciated that there are a number of other antibacterial compounds that also derive their activity through interference with bacterial peptidoglycan synthesis and/or crosslinking. Several of these have available primary amines that could be modified in a synthesis protocol as described here, for example, natural, synthetic, or semi-synthetic glycopeptide antibiotics (teicoplanin, ramoplanin, decaplanin, oritavancin, dalbavancin, ramoplanin, THRX-1179 (see FIG. 8), etc.), bacitracin, cephalexin, cefadroxil, cefaclor, cefotaxime, cefprozil, loracarbef, ceforanide, cefepime, cefibutin, cefdinir, cefditorin pivoxel, ceftizoxime, ceftazidime, ceftriaxone, ceftazidime, cephradine, cefixime, aztreonam, amoxicillin, penicillamine, and ampicillin. The enzyme lysostaphin or antimicrobial peptides could also be functionalized in place of a conventional antibiotic in a completely analogous fashion.

It should be noted that antibacterial polymers of the type described will be quite efficacious against gram positive organisms. Since gram negative bacteria have a much decreased peptidoglycan content, many antibacterials that target these organisms require internalization, though not all (e.g. aztreonam, amoxicillin, ampicillin, second and third generation cephalosporins, and antibacterial peptides). It should be appreciated that by choosing an appropriate antibiotic, one can also construct surfaces that kill gram negative bacteria as well.

Appropriate polymers can also be designed to release the antibiotic compound. This can be accomplished by incorporating degradable functionalities into the starting monomers. Suitable degradable functionalities include, but are not limited to, poly(lactic), poly(glycolic), or poly(lactic-co-glycolic) acid segments if hydrolytic cleavage is desired. One can also utilize enzymatically degradable functional groups.

The photopolymerization process offers a very convenient means of applying the coating and potentially offers a means of simultaneously sterilizing implant materials, and ultimately affords control over polymer properties. In addition, a living-radical type silane can also be used on the implant surface and thus forego the need for a solution-based initiator. Such a silane allows for a relatively more precise control of coating architecture including well-defined polymer patterning, control of backbone chain length, and elimination of bulk polymerization. One can also use this approach to construct useful block copolymers of different polymerizable antibacterials or other polymerizable bioactive species. In this manner, known synergistic effects between biomolecules can be thus exploited.

Compositions and methods of the invention also allow loading of VPA-type coatings with bioactive species for elution purposes. For example, surface coated polymers of the invention can be loaded with growth factors or other proteins, antibiotics, drug-loaded microparticles, and the like.

Compositions (e.g., VPA-like compounds) and methods of the invention are also useful for incorporating into existing bone cement formulations. Compositions of the invention covalently polymerize into the matrix of such bone cement and serve the purpose of offering long-term resistance to bacterial colonization that traditional loading of antibiotics cannot afford. Furthermore, since antimicrobial compounds become chemically part of the bone cement matrix—e.g., VPA acrylate functionalities readily react with bone cement methacrylates—and are not simply physically entrapped, they are expected to greatly decrease the problems associated with conventional methods for adding antimicrobial compounds to cement formulations.

Thus, a surgeon can choose to use a VPA-containing bone cement when implanting a cemented total hip replacement, or he could use a VPA-coated uncemented prosthesis. Infection can also present challenges in association with indwelling medical devices beyond the sphere of orthopaedic hardware. Urinary catheters, IV lines, cardiac catheters, vascular catheters, pacemakers, stents, vascular grafts, and nasogastric tubes all incorporate some form of polymeric material that may become colonized with undesirable organisms. There have been numerous attempts to load the surfaces of these various devices with antibiotic compounds for subsequent elution. In general, however, it has been difficult to adequately control the release of antibiotics, thus rendering the coatings only temporarily effective.

Methods and compositions of the invention allow VPA-like compounds to be easily polymerized into numerous polymer-type medical devices made from materials like poly(ethylene), poly(proplylene), poly(vinyl chloride), polystyrene, as well as latex or silicone rubber, thus providing covalently attached, surface active antimicrobial (e.g., antibacterial) compounds.

Moreover, methods and compositions of the invention also provide means for combining antimicrobial (e.g., VPA-like antibacterial) compounds with other acrylated biomolecules. For example, VPA can be copolymerized with an acrylated form of bone-morphogenetic-protein-2 (BMP-2) to produce a polymer coating having antibacterial properties as well as bone inductive ones. Such combination is useful, for example, for applying to the femoral component of a prosthetic hip replacement to prevent and/or treat infection while simultaneously promoting bone ingrowth.

Compositions and methods of the invention can be: (1) polymerized to/from surfaces including metallic implants by photochemical means (living radical or otherwise); (2) covalently incorporated into traditional poly(methy methacrylate) bone cements or any other polymerizable bone cement (without a deleterious effect on mechanics) where surface-active antibiotics are desired; (3) copolymerized with various monomers, cross-linkable or otherwise, to form spatially complex constructs useful for tissue repair or coating applications and that these constructs/coatings can be loaded with other elutable biomolecules when desired; (4) copolymerized as part of polymer-type medical devices including, but not limited to, catheters, IV tubing, and vascular stents, thereby rendering these devices bacterial resistant; and/or (5) copolymerized with polymerizable growth factor-type molecules (e.g. acrylated proteins) to fight infection as well as influence cell growth and/or differentiation.

The present invention will be described with regard to the accompanying drawings and the following examples which assist in illustrating various features of the invention. In this regard, the present invention generally relates to a polymerizable antimicrobial composition and methods for using the same.

In one aspect, the polymerizable antimicrobial composition of the invention comprises an antimicrobial compound, a linker, and a polymerizable functional group. In some embodiments, the polymerizable functional group is an olefin. The antimicrobial compound refers to any compound that kills, inhibits, or reduces the number of microorganisms such as bacteria, virus, yeast, and other single cell organisms. Typical antimicrobial compounds include, but are not limited to, antibacterial compounds, antiviral compounds, anti-yeast compounds, as well as other antimicrobial compounds known to one skilled in the art. In one embodiment, the antimicrobial compounds of the invention are those approved by the Federal Drug Administration (FDA) for treatment of microbial infections. Within this embodiment, in some instances the antimicrobial compound is an antibacterial compound.

In some instances, the antibiotic vancomycin was modified so that it can be converted into a polymerizable monomer. In particular, a poly(ethylene glycol)-monoacrylate form of vancomycin was synthesized. It is believed that the PEG linker provides mobility to the vancomycin molecule once a polymer has been formed. The acrylate functionality provides unsaturated carbon-carbon double bonds available for free-radical polymerization. The acrylate functionality can be polymerized (e.g., photochemically or by other suitable polymerization reaction) to the surfaces of medical devices such as titanium alloy orthopaedic implants, following appropriate surface preparation. The medical device surface so coated is believed to be bacteriostatic or bactericidal to gram positive microorganisms such as Staphylococcus aureus and Staphylococcus epidermidis, which are the most common causes of orthopaedic infections. The resulting polymer-coated surfaces are also believed to be resistance to biofilm colonization. The resulting coatings are physically stable and have multiple, active vancomycin molecules pendant to each polymer chain. Accordingly, compositions and methods of the invention allow a significantly larger amount of antimicrobial compound to be present at the implant-tissue interface when compared to monolayer coverage systems.

The polymerizable form of vancomycin can also be directly polymerized into the matrix of existing poly(methyl methacrylate) bone cements, thus making them resistant to biofilm colonization without compromising mechanical properties.

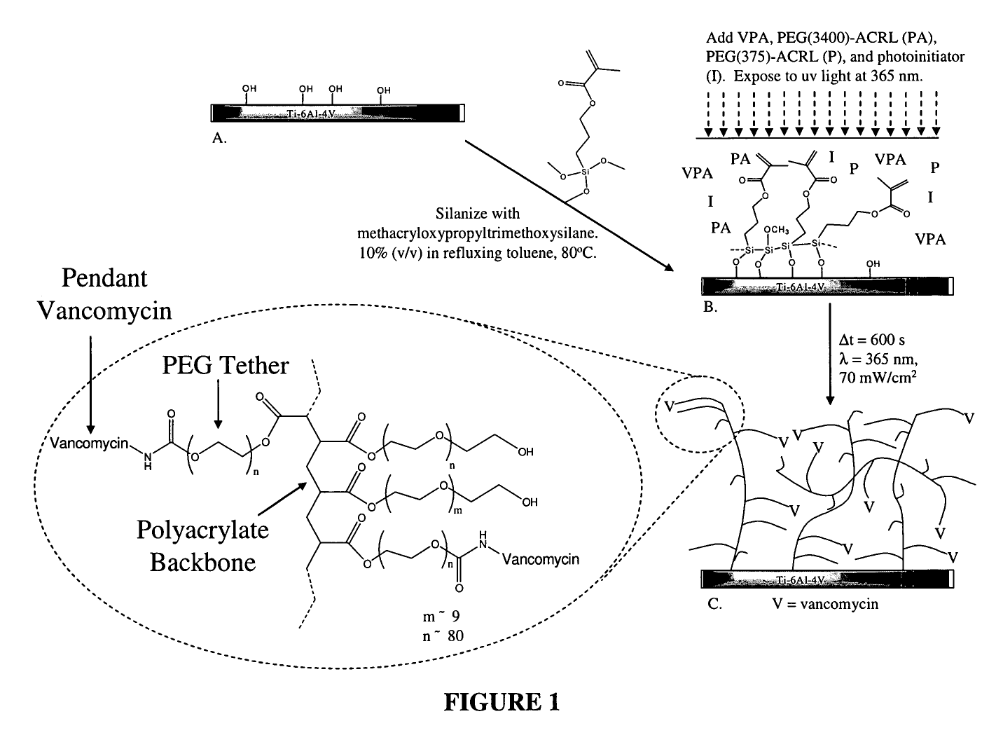

One particular general scheme is illustrated in FIG. 1. As shown in FIG. 1, a titanium alloy surface is silanized with a species having reactive end groups (methacrylates). The acrylated drug Vancomycin-PEG(3400)-acrylate is then photochemically grafted to/from the silanized surface. The resulting titanium alloy surface has covalently attached polyacrylate polymer chains with pendant, pegylated vancomycin molecules. In particular, FIG. 1 illustrates a photochemical attachment of VPA to silanized Ti-6Al-4V Alloy. As shown in step A, oxidized Ti-6Al-4V alloy was modified with methacryloxypropyltrimethoxysilane to produce a surface having photo-reactive methacrylate functionalities (step B). A VPA-PEG(3400)-monoacrylate-PEG(375)-monoacrylate copolymer coating was photopolymerized to the surface. As shown in step C, the resulting coating has both bulk-polymerized and surface-bonded polyacrylate polymer chains with pendant, pegylated vancomycin, pendant PEG(375), and pendant PEG(3400). This allows for 3-D complexity and for a coating with orders of magnitude more vancomycin than an analogous monolayer system.

Infections in the setting of orthopaedic hardware remain a serious complication. Traditional treatment modalities rely on antibiotic-loaded biomaterials and/or prolonged intravenous therapy, both of which suffer from major limitations. The present inventors have found that a derivatized form of the glycopeptide antibiotic vancomycin can be covalently attached to a Ti-6Al-4V implant alloy to form a bactericidal surface capable of reducing the frequency (probability or a number of instances) of orthopaedic infections, for example, by killing bacteria that are responsible for the infection.

As discussed in detail in the Examples section, a polymerizable poly(ethylene glycol)-acrylate derivative of vancomycin was synthesized. The monomer was subsequently photochemically polymerized to implant grade Ti-6Al-4V alloy. The coating was bactericidal against Staphylococcus epidermidis. Without being bound by any theory, it is believed that such antibacterial activity is achieved in some instances through release of unattached antibiotic species followed by continued surface-contact-mediated bacterial killing by covalently tethered vancomycin. In some embodiments, the number of colony forming units can be reduced to about one-half or less (often about one-fifth or less and more often about one-tenth or less) from an initial inoculum concentration (e.g., 1×10⁶ cfu/mL over 4 hours). And in some instances the number of cfu can be reduced by about one-tenth or less (often one-fiftieth or less and more often about one-hundredth or less) with respect to nonbactericidal control surfaces.

In some instances, an inoculum of 1×10⁴ cfu/mL was reduced to substantially undetectable levels over 17 hours. Accordingly, methods of the invention allow a loading dose several thousand times larger than that achieved with monolayer vancomycin coupling methods.

Infection following the implantation of orthopaedic hardware remains a serious and expensive complication. More than 1 million hip replacements are performed each year worldwide and modern surgical practice has reduced the incidence of infection following total hip arthroplasty to approximately 1% to 2% in the general population. However, those patients with periprosthetic joint infection must undergo lengthy antibiotic therapy and quite often surgical revision. Two-stage operations in which contaminated hardware is removed and temporarily replaced with antibiotic-loaded spacers or beads are common. The physiologic location of orthopaedic infections makes them difficult to treat due to poor antibiotic penetration into bone and joint spaces and the formation of bacterial biofilms. Accordingly, extensive wound irrigation and debridement at the time of surgery is needed to reduce the number of infection incidences. Furthermore, recurrent infection may be problematic, even in patients with competent innate and adaptive immune responses. Problems including loss of viable bone stock, long periods of hospitalization, severe functional impairment, and less than ideal treatment options present challenges to both surgeon and patient. Methods of the invention avoid or reduce such risk.

Various materials for treating and preventing orthopaedic infections are conventionally known. Many conventional methods involve novel biomaterials and ways of loading those materials with pharmaceuticals. One such example is antibiotic-loaded poly(methyl methacrylate) bone cement, which is quite effective at delivering high local levels of an antibacterial agent for a short period of time, usually a few days. When loaded at high dose (>2 g antibiotic/40 g cement), antibiotic-loaded bone cement may provide effective antibiotic levels in surrounding tissues for more extended periods but mechanical properties of the cement may be compromised.

The present inventor have found that in some instances a derivatized form of the glycopeptide antibiotic vancomycin can be covalently attached to a Ti-6Al-4V implant alloy using a technique that exploits the functionalization of antibiotics with polymerizable moieties, thus forming an activated surface capable of possessing antibacterial property to reduce orthopaedic infections.

Additional objects, advantages, and novel features of this invention will become apparent to those skilled in the art upon examination of the following examples thereof, which are not intended to be limiting.

### EXAMPLES

### Materials and Methods

Polymerizable vancomycin monomer, vancomycin-PEG(3400)-acrylate (VPA), was produced, purified (e.g., by gel filtration chromatography), and characterized (e.g., by NMR spectroscopy). To statistically evaluate biological changes in monomer activity, the Minimum Inhibitory Concentration (MIC) and Minimum Bactericidal Concentration (MBC) values for the new species were determined. In addition, a statistical comparison of the surface-based antibacterial activity of polymerized VPA with respect to various controls was made. All MIC and MBC experiments were conducted in triplicate, and surface challenge studies were conducted in at least triplicate.

### Synthesis of Vancomycin-PEG(3400)-acrylate

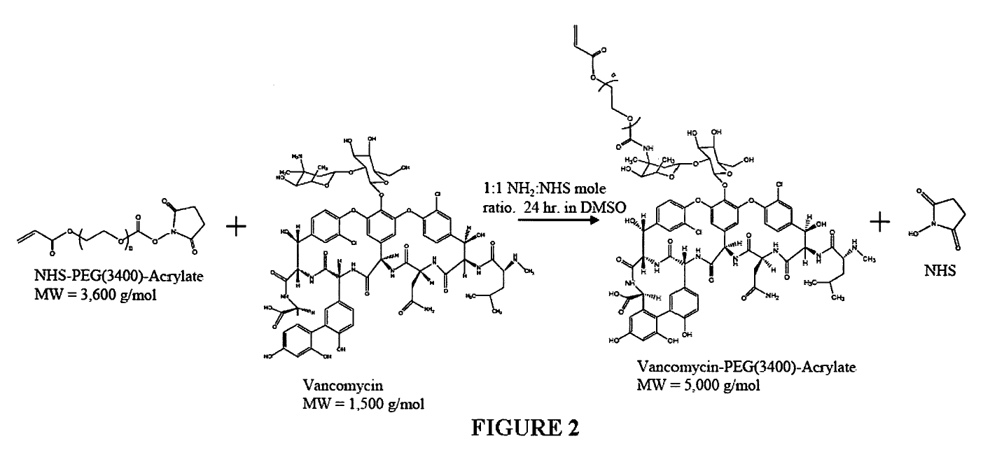

Vancomycin-PEG(3400)-acrylate (VPA) was synthesized by reaction of vancomycin hydrochloride (Sigma Aldrich, St. Louis, Mo.) with the pegylation reagent N-hydroxysuccinimide-PEG(3400)-acrylate (NHS-PEG(3400)-acrylate) (Nektar Therapeutics, San Carlos, Calif.) in a 1:1 mole ratio in DMSO at 25° C. for 24 hr. (FIG. 2). The 3400 denotes the approximate molecular weight in Daltons of the PEG spacer. The pegylation reagent reacts with the primary amine at what has been termed the V₃ position of vancomycin to form a single-adduct product. See FIG. 2.

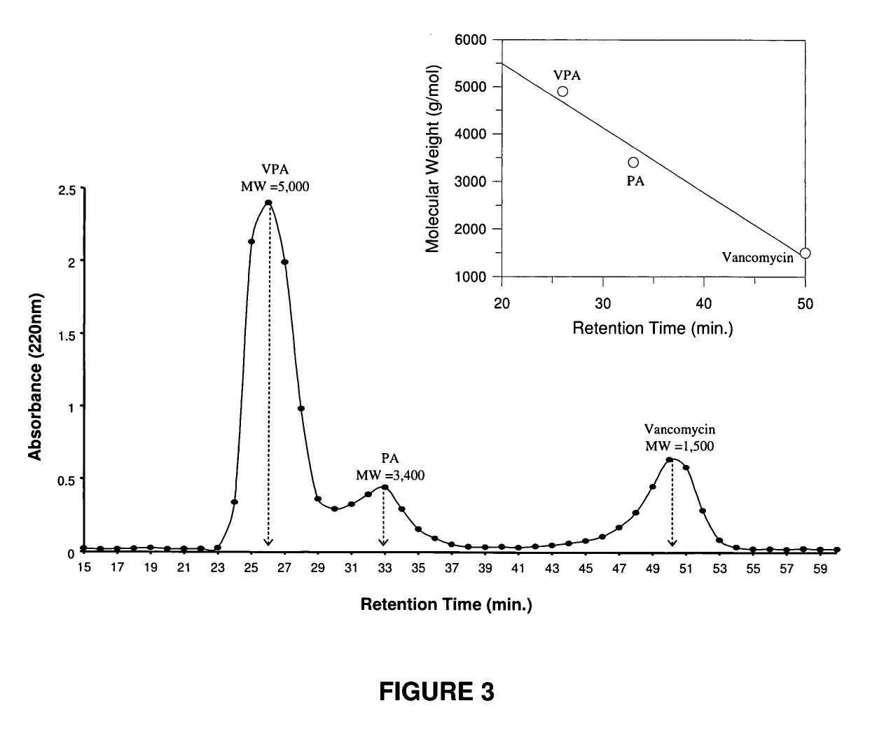

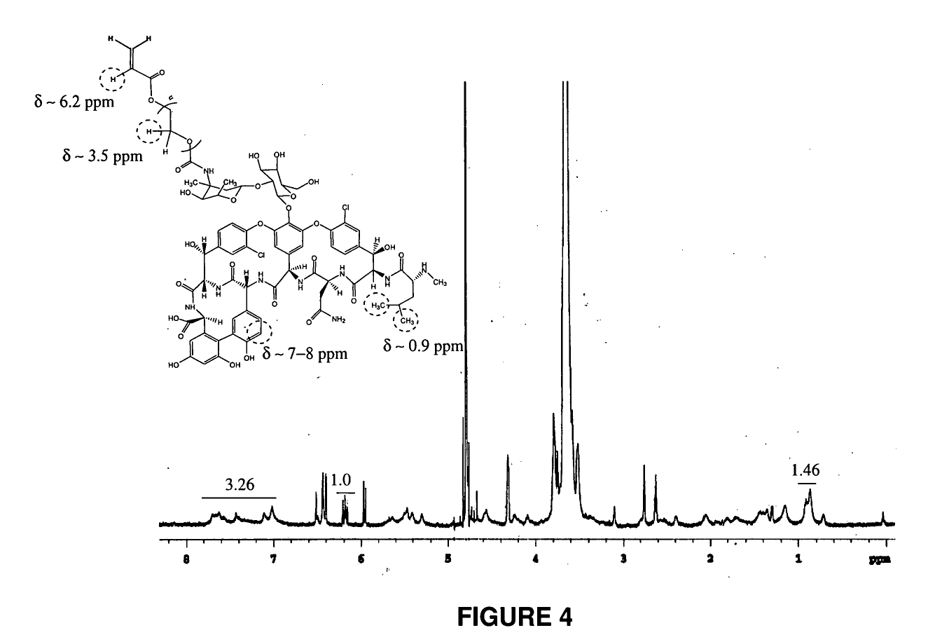

The product was purified by gel filtration chromatography on a Sephadex G-25F (Amersham Biosciences, Uppsala, Sweden) column with dimensions 33 cm×2.5 cm (height×inside diameter) with deionized H₂O as the mobile phase (3 mL/min volume flow rate). The product was recovered by spectrophotometric analysis of column breakthrough at 220 nm using a Lambda 40 UV/vis spectrophotometer (PerkinElmer Instruments, Wellesley, Mass.). See FIG. 3, which shows VPA elutes first followed by PEG(375)-monoacrylate and vancomycin. Briefly, DMSO was removed on a column first pass. On second pass, reactants were separated from product. The highest molecular weight species eluted first. As shown in FIG. 3, VPA elutes at ca. 26 min., PEG(3400)-acrylate at ca. 33 min., and vancomycin at ca. 50 min. These results suggest high reaction efficiency. The results also suggest a single-adduct product was recovered since all species greater than ca. 5,000 molecular weight would be expected to be washed out with the void volume (size exclusion limit ca. 5,000 for Sephadex G-25). Appropriate fractions were collected, frozen at −80° C., and lyophilized for 72 hr. on a Freezone 4.5 lyophilizer (Labconco, Kansas City, Mo.). The newly formed monomer's chemical structure was characterized by 500 MHz ¹H NMR spectroscopy (see FIG. 4). ¹H NMR spectroscopy of recovered VPA showed the expected chemical shifts and approximately 25% acrylated product based on peak integrations. Thus, the recovered product contained 25% VPA and 75% PEG(3400)-monoacrylate.

### MIC and MBC of Vancomycin-PEG(3400)-acrylate

The minimum inhibitory concentration (MIC) and minimum bactericidal concentration (MBC) of the monomer were determined by the broth dilution method. Various amounts of VPA were added to 1 mL of brain heart infusion media (BHI) (Becton, Dickinson, and Co., Sparks, Md.) along with 1×10⁵ cfu of S. epidermidis ATCC 12228 (MicroBiologics, Inc., St. Cloud, Minn.) using aseptic technique. Cultures were incubated for 24 hr. at 37° C. The MIC was taken to be the lowest VPA concentration at which no turbidity was observed. Based on this test the MIC was determined to be 11.5±0.8 μg/mL (mean ±std. dev., n=3).

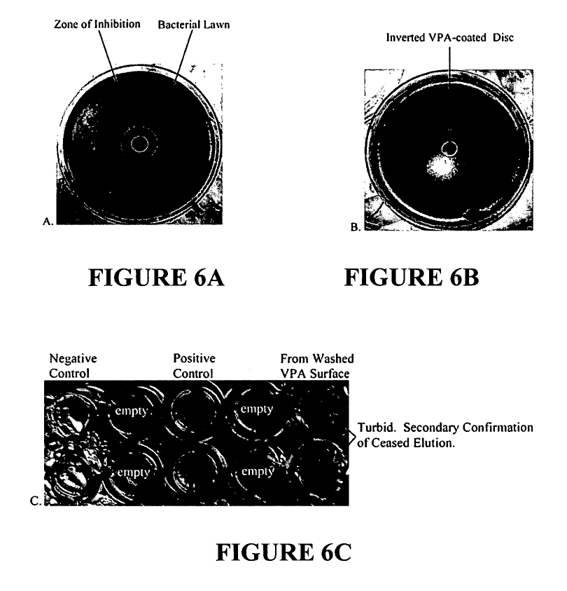

The MBC is another parameter used to assess the usefulness of an antibacterial. It provides some measure of the concentration of agent required to kill bacteria, not just inhibit their growth. In this test, 10 μL of media from each tube were plated on trypticase soy agar (TSA) plates with 5% sheep blood (Becton, Dickinson, and Co., Sparks, Md.) and grown for 24 hr. at 37° C. The MBC was taken to be the lowest VPA concentration at which no colonies were observed. FIG. 6 shows plates obtained in the MBC determination of VPA against S. epidermis ATCC 12228. The MBC was found to be 15±1 μg/mL or, equivalently, 3.0±0.2 μM (mean ±std. dev., n=2).

### Surface Coating

Ti-6Al-4V alloy discs were machined from surgical grade bar-stock to dimensions 10 mm×3 mm (d×h). Surfaces were cleaned by 1 hr. sonication in acetone followed by 1 hr. sonication in distilled water. Discs were oxidized 12 at a time by treatment with a solution of 25 mL of 98% sulfuric acid and 25 mL of 35% hydrogen peroxide. Discs were then rinsed in distilled water, dried at 80° C. overnight, and examined by scanning electron microscopy (SEM) without gold coating. Oxidation of Ti-6Al-4V discs was assessed by SEM and x-ray photoelectron spectroscopy (XPS, data not shown). Oxidized discs were visibly dulled, and SEM images showed that the oxidation protocol etched the surface, both suggesting increased oxide content.

In order to provide a surface capable of free-radical polymerization with VPA, oxidized Ti-6Al-4V discs were silanized with methacryloxypropyltrimethoxysilane to give a monolayer with reactive methacrylate groups to or from which polymer chains can be polymerized as illustrated in FIG. 1. The process of silanization involved reacting a silane—a species analogous to a hydrocarbon in which the backbone carbons have been replaced with silicone atoms—with surface hydroxyl groups in order to “prime” the surface for further chemical modification. Briefly, twelve discs were added to a reflux apparatus with 10% (v/v) methacryloxypropyltrimethoxysilane (Gelest, Inc., Morrisville, Pa.) in anhydrous toluene (Acros, Geel, Belgium) and stirred for 4 hr. at 80° C. under argon atmosphere. Discs were removed, rinsed with anhydrous toluene, and air dried for later use. Oxidized and silanized surfaces were characterized by XPS with a 45° incident, monochromatic x-ray beam (data not shown).

The oxidation procedure appeared to have increased the thickness of the oxide layer beyond that of the native oxide as demonstrated by the increased oxygen signal and attenuated Ti 2p signal. There also appeared to be a change in the structure of the Ti 2p peak, suggesting a change in electronic environment as would occur in the oxide versus the metal. XPS also showed (data not shown) that the silanization procedure deposited silicon on the alloy surface as demonstrated by the further attenuated titanium signal and appearance of silicon signals.

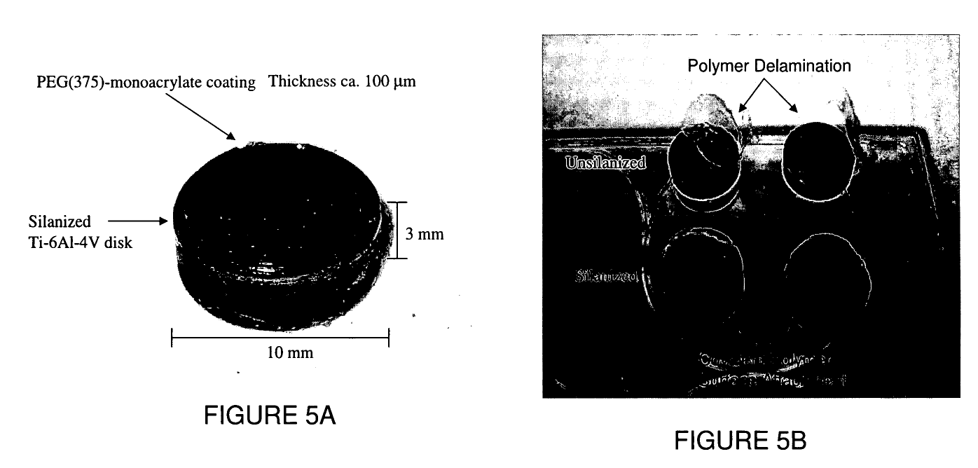

To form an antibacterial grafting solution for subsequent photochemical polymerization, 80 μL of VPA monomer at a concentration of 333 mg/mL in Ar-purged DMSO were mixed with 100 μL of the photoinitiator 1,1-dimethoxy-1-phenyl acetophenone (DMPA) at a concentration of 2 mg/mL in Ar-purged DMSO, 50 μL of PEG(375)-monoacrylate (Sigma Aldrich, St. Louis, Mo.), and 20 μL of Ar-purged DMSO. Surfaces were grafted by photopolymerization of 15 μL of this co-monomer solution at 365 nm from a 70 mW/cm² light source provided by a Hybralign Series 200 mask alignment system (Oriel Instruments, Stratford, Conn.). This formulation provided for an approximately 1:100 mole ratio of VPA to the sum of other copolymer species in the coating. It should be noted that these concentrations may be varied as desired to increase or decrease the VPA surface density and that mono- or multi-functional monomers other than PEG(375)-monoacrylate can be used as copolymers. The polymerization process led to a covalently bound coating with a thickness dependent upon the volume of the monomer solution applied. A substantially uniform coatings were formed with a thickness that was ca. 100 μm (see FIG. 5A). Stability of the modified surface depended on the silanization and functionalization of the titanium surface with a polymerizable group (e.g., methacrylate) (see FIG. 5B). PEG(375)-monoacrylate surfaces were prepared in an analogous fashion by substituting Ar-purged DMSO for VPA/PEG(3400)-monoacrylate. These were used as negative controls for subsequent biological coating characterization.

One of the purposes of the silane monolayer was to provide a means of covalently attaching antibacterial polymer to the Ti-6Al-4V surface. To assess the ability of silanized surfaces to bond polymers directly to titanium alloy, delamination experiments were conducted. After a 15 minute soak in methanol, polymer films on non-silanized surfaces completely delaminated. Films on silanized surfaces did not delaminate in methanol during a 48 hr. exposure. Films on silanized surfaces were resistant to delamination for one week plus in deionized H₂O. As shown in FIG. 5B, silanization prevents polymer delamination. In FIG. 5B, discs in the back have no silane monolayer, and the discs in front do.

Extended washes were employed to elute unreacted monomer and loosely entangled polymer chains from VPA copolymer-coated discs. A variation of the Kirby-Bauer disc diffusion assay was implemented to temporally define this elution. Polymer-coated discs having undergone a brief surface rinse or a 3-day wash—24 hours in 10 mL deionized H₂O, 24 hours in 10 mL EtOH, and 24 hours in 10 mL deionized H₂O—were inverted on trypticase soy agar (5% sheep blood) plates having been spread with 450 μL of 2×10⁷ cfu/mL S. epidermidis ATCC 12228. Plates were cultured for 24 hours at 37° C. The presence of a zone of inhibition was indicative of elution of untethered vancomyin.

An additional test for antibacterial elution was employed by incubating 100 μL of brain heart infusion media on polymer surfaces for 4 hours or 17 hours, collecting 50 μL of that media, loop inoculating with S. epidermidis ATCC 12228, and culturing for 24 hours at 37° C. Turbidity was considered a secondary confirmation that elution had measurably stopped.

Once elution had measurably stopped, the surface-contact-mediated antibacterial action was evaluated. S. epidermidis ATCC 12228 was suspended in brain heart infusion media, and either 1×10⁵ cfu or 1×10³ cfu in 100 μL were allowed to settle onto the polymer-coated surfaces. Following 4-hour or 17-hour incubations, respectively, the suspensions were sampled, serially diluted with deionized water, and plated on trypticase soy agar (5% sheep blood) for cfu counts.

All statistical analysis was performed using Minitab15 statistical software (Minitab, Inc., State College, Pa.). For MIC and MBC comparisons, the Mann-Whitney test was used. This is a nonparametric test that makes no assumption about an underlying distribution (e.g. no normality assumption) and is appropriate for comparing discrete data (i.e. predetermined to a set of values) such as MIC and MBC values. A p<0.1 was considered significant. Comparisons of surface antibacterial activity were done using the one-way ANOVA in conjunction with the Tukey-Kramer Procedure for the comparison of treatment means. ANOVA p<0.1 was considered significant. Levene's Test was used to check the assumption of equal sample variances, and the Kolmogorov-Smimov Test along with visual examination of normal probability plots was used to verify data normality. In general, the null hypothesis of equal sample variances could be rejected for surface challenge cfu/mL data (all p<0.100), so cfu/mL data were logarithmically transformed and then re-evaluated using Levene's Test. The null hypothesis of equal sample variance for the transformed data could not be rejected (all p>0.846). The null hypothesis of normality for the transformed (and non-transformed) data was not rejected by the Kolmogorov-Smimov Test (reported in Minitab as all “p>0.150”). The one-way ANOVA was then used to evaluate the transformed data. ANOVA p values were found to be similar before and after transformation, suggesting ANOVA robustness to unequal variances in our data. Thus, when it was not possible to perform a logarithmic transformation (i.e. for data sets containing replicates where no bacteria were detected), the ANOVA was conducted without data transformation.

Preliminary experiments were also conducted to examine the anti-biofilm properties of the VPA coating. A Ti-6Al-4V disc, a three-day washed VPA-coated disc (cografted with PEG(375)-monoacrylate), and a PEG(375)-monoacrylate coated disc were each placed in individual 125 mL Erlenmeyer flasks with 50 mL of sterile BHI media using aseptic technique. The media was loop inoculated with S. epidermidis ATCC 35984 (ATCC, Manassas, Va.), a clinical isolate and prototypical biofilm producer. The flasks were placed on a rotary shaker (110 rpm) at 37° C., media was exchanged every 24 hr., and biofilm growth was visually evaluated at 24 hr. intervals. S. epidermidis had little difficulty forming a biofilm on Ti-6Al-4V alloy. However, both VPA-coated and PEG(375)-monoacrylate coated surfaces significantly retard biofilm adherence.

### Results

To demonstrate surface-based activity of photopolymerized VPA, it was deemed necessary to show bacterial killing in the absence of biologically significant elution of antibacterial species. Results from the bioassays used for this purpose are provided below (FIG. 6). In particular, FIG. 6A shows a well-defined zone of inhibition around a coated disc (inverted) having undergone no extended wash steps is indicative of antibacterial elution; Figure B shows where there was a 3-day series of washes, there is no visible zone of inhibition, indicating that the wash steps were sufficient to elute unreacted VPA or loosely entangled polymer chains; and FIG. 6C shows wells from a 96-well culture plate containing either clear brain heart infusion media or turbid BHI media. The negative control was uninoculated. The positive control was loop-inoculated with S. epidermidis ATCC 12228. Media that was exposed (4 hours and 17 hours) to a 3-day-washed VPA copolymer coating and loop inoculated with bacteria was turbid as well. This was considered further confirmation that antibacterial elution had stopped.

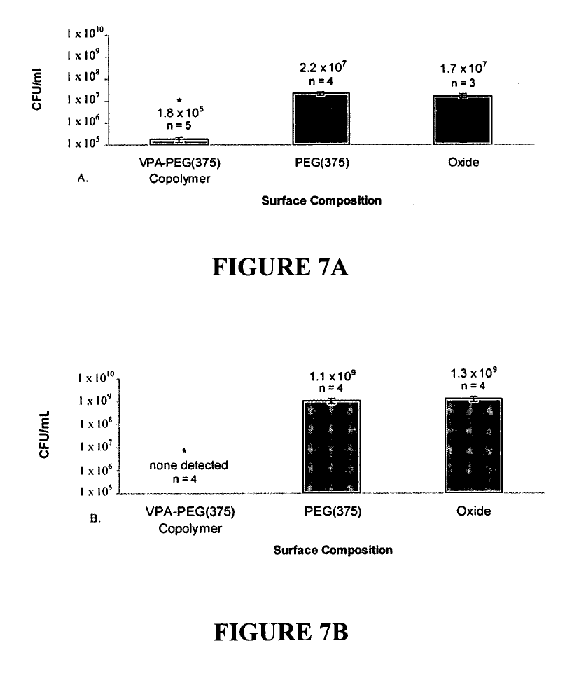

VPA-coated Ti-6Al-4V discs exhibited surface-contact-mediated antibacterial action after elution of active species had measurably stopped. After a 4-hour bacterial incubation, VPA-coated surfaces (cografted with PEG(3400)-monoacrylate and PEG(375)-monoacrylate) reduced viable bacterial levels by about five-fold from the initial inoculum of 1×10⁶ cfu/mL. Bacteria in contact with either PEG(375)-monoacrylate or oxidized Ti-6Al-4V surfaces significantly increased in number by some 100-fold over the VPA-exposed organisms (p<0.001 with respect to either control). See FIGS. 7A and 7B, which shows antibacterial surface activity of VPA copolymer coating. Surface-mediated killing of S. epidermidis was seen after elution had measurably stopped. These results demonstrate the feasibility of photopolymerizing antibacterial monomers to implant materials to yield bactericidal surfaces. FIG. 7A shows the result of an inoculum of 1×10⁶ cfu/mL S. epidermidis ATCC 12228 that was incubated in contact with 3-day-washed surfaces for 4 hours. The VPA-PEG(3400)-monoacrylate-PEG(375)-monoacrylate copolymer significantly reduced the bacterial load (p<0.001 with respect to either control). FIG. 7B shows the result of an inoculum of 1×10⁴ cfu/mL S. epidermidis ATCC 12228 that was incubated in contact with 3-day-washed surfaces for 17 hours. No bacteria were detected in the presence of the VPA-type copolymer. The bacterial reduction was significant with respect to either control surface (p<0.001). Accordingly, after a 17-hour bacterial incubation, VPA-type surfaces reduced viable bacterial levels of an initial inoculum of 1×10⁴ cfu/mL to undetectable levels, which was again significantly less than bacterial counts from control surfaces (p<0.001 with respect to either control).

Consistent with the synthesis protocol, three peaks were observed by gel filtration chromatography with species eluting in order of decreasing molecular weight: VPA eluted at ca. 26 minutes, hydrolyzed NHS-PEG(3400)-acrylate at ca. 33 minutes, and vancomycin at ca. 50 minutes (see FIG. 2). Furthermore, ¹H NMR characterization of the VPA fraction demonstrated the expected functionalities of vancomycin-PEG(3400)-acrylate: vinyl hydrogens from the acrylate functionality are apparent at a chemical shift of 6 to 6.5 ppm; PEG hydrogens are apparent at a chemical shift of 3.5 to 4 ppm; aromatic hydrogens from vancomycin are apparent at 7 to 7.8 ppm; and methyl hydrogens from vancomycin are apparent at 0.9 ppm. The methyl integration of 1.46 (FIG. 3) indicates approximately 25% acrylated product and 75% PEG(3400)-acrylate. This estimate is further confirmed by the aromatic hydrogen integration of 3.26 (13 aromatic hydrogens are expected for 100% VPA).

The VPA monomer had a MIC greater than that of vancomycin (p=0.08) against S. epidermidis ATCC 12228. The VPA MBC may also be greater than the VPA MIC (p=0.15). The VPA monomer has a MIC of 11.5±0.8 μg/mL or, equivalently, 2.35±0.15 μM. The MBC for VPA is 15±1 μg/mL or, equivalently, 3.0±0.2 μM. The MIC and MBC of pure vancomycin were found to be 1.12±0.04 μM. VPA retained activity at a low molar concentration.

Without being bound by any theory, it is believed that most of the coating mass is covalently bound to the titanium substrate. However, it appears the VPA-coated Ti-6Al-4V discs eluted a small fraction of either unreacted VPA monomer or unbound polymer chains at detectable levels for approximately 3 days. A modified Kirby-Bauer disc diffusion assay, as described previously, was used to detect elution of active products. After 24 hour incubation, a clear zone of inhibition was apparent around the inverted unwashed disc (FIG. 6A). A disc that underwent three consecutive wash steps showed no zone of inhibition (FIG. 6B), suggesting that the elution had stopped. To confirm this observation, 3-day washed VPA-coated surfaces were exposed to brain heart infusion media, and the media was tested for antibacterial activity. No activity was detected after a 4-hour or 17 hr surface exposure (FIG. 6C), further suggesting elution had dropped to undetectable levels.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed:

1. An antimicrobial composition of the formula:
V-L-A
wherein
V is a moiety comprising a polymerizable functional group;
L is a linker comprising from about 5 to about 500 linking chain atoms; and
A is an antimicrobial compound.

2. The antimicrobial composition of claim 1, wherein the antimicrobial compound is an antibacterial compound.

3. The antimicrobial composition of claim 2, wherein the antibacterial compound is a glycopeptide antibiotic, bacitracin, cephalexin, cefadroxil, cefaclor, cefotaxime, cefprozil, loracarbef, ceforanide, cefepime, cefibutin, cefdinir, cefditorin pivoxel, ceftizoxime, ceftazidime, ceftriaxone, ceftazidime, cephradine, cefixime, aztreonam, amoxicillin, penicillamine, ampicillin, an antibiotic enzyme, or a combination of two or more thereof.

4. The antimicrobial composition of claim 3, wherein the glycopeptide antibiotic is natural, synthetic, or semi-synthetic glycopeptide.

5. The antimicrobial composition of claim 4, wherein the glycopeptide antibiotic is vancomycin, teicoplanin, ramoplanin, decaplanin, oritavancin, dalbavancin, ramoplanin, or THRX-1179.

6. The antimicrobial composition of claim 3, wherein the antibiotic enzyme is lysostaphin.

7. The antimicrobial composition of claim 1, wherein A is linked to L via an amide bond, an ester linkage, a sulfide linkage, a disulfide linkage, a thiosuccinimidyl ether linkage, a thiosulfonyl linkage, a urethane linkage, a urea linkage, a secondary amine linkage, or a combination thereof.

8. The antimicrobial composition of claim 1, wherein L is polyethylene glycol, polylactic acid, polyglycolic acid, poly(lactic-co-glycolic) acid, polyurethane, polyester, polypeptide, or a combination thereof.

9. The antimicrobial composition of claim 1, wherein the antimicrobial compound is cleavable from the linker in vivo.

10. A method for producing a substrate comprising a covalently linked antimicrobial composition comprising:
providing a substrate comprising a reactive functional group; and
contacting an antimicrobial composition of the formula:
V-L-A
wherein
V is a moiety comprising a polymerizable functional group;
L is a linker comprising from about 5 to about 500 linking chain atoms; and
A is an antimicrobial compound,
under conditions sufficient to form a bond between the reactive functional group on the substrate and the polymerizable functional group of the antimicrobial composition to produce the substrate comprising a covalently linked antimicrobial composition.

11. The method of claim 10, wherein the polymerizable functional group is an olefin.

12. The method of claim 11, wherein said step of forming a bond between the reactive functional group of the substrate and the polymerizable functional group of the antimicrobial composition comprises photopolymerization.

13. The method of claim 11, wherein said step of forming a bond between the reactive functional group of the substrate and the polymerizable functional group of the antimicrobial composition comprises radical polymerization.

14. The method of claim 13, wherein the radical polymerization is initiated by a thermal activatable initiator, a visible light or long wavelength ultraviolet light-activatable initiator, benzoyl peroxide, potassium persulfate, ammonium persulfate, or other free-radical initiator.

15. The method of claim 11, wherein said step of forming a bond between the reactive functional group of the substrate and the polymerizable functional group of the antimicrobial composition comprises living radical polymerization.

16. The method of claim 10, wherein said step of forming a bond between the reactive functional group of the substrate and the polymerizable functional group of the antimicrobial composition further comprises encapsulating a bioactive material within a polymer matrix that is formed on the substrate.

17. The method of claim 10, wherein the substrate is a medical product.

18. A medical product comprising a covalently linked antimicrobial composition of claim 1, wherein the covalently linkage is formed from a reaction between the polymerizable function group that is present on the moiety V and a functional group that is present on the medical product.

19. The medical product of claim 18, wherein said medical product is bone cement, tissue adhesive, polymeric bone graft, synthetic tissue scaffold, wound dressing, tissue adhesive, a medical device, a dental composite, or an orthopaedic hardware.

20. The medical product of claim 19, wherein said medical device is a catheter, an intravenous catheter or line, a central line, a stent, a vascular graft, a nasogastric tube, a polymeric suture, or a contact lens.
